(19) United States
(12) Patent Application Publication
(10) Pub. No.: US 2021/0327579 A1
(43) Pub. Date: Oct. 21, 2021

(54) METHOD AND APPARATUS FOR CLASSIFYING SUBJECTS BASED ON TIME SERIES PHENOTYPIC DATA

(71) Applicant: Oxford University Innovation Limited, Oxford (GB)

(72) Inventors: Andrew David CLIFTON, Oxford (Oxfordshire) (GB); Nazli FARAJIDAVAR, Oxford (Oxfordshire) (GB); Tingting ZHU, Oxford (Oxfordshire) (GB); Xiaorong DING, Oxford (Oxfordshire) (GB); Peter WATKINSON, Oxford (Oxfordshire) (GB)

(21) Appl. No.: 17/051,796

(22) PCT Filed: Mar. 12, 2019
(86) PCT No.: PCT/GB2019/050683

§ 371 Date: Oct. 30, 2020

(30) Foreign Application Priority Data
May 3, 2018 (GB) 1807307.2

(51) Int. Cl. G16H 50/20; G06N 20/00; G06N 7/00; G16H 10/60; G16H 50/70; A61B 5/0205; A61B 5/021; A61B 5/024; A61B 5/08; A61B 5/145; A61B 5/16; A61B 5/00

(52) U.S. Cl. CPC G16H 50/20; G06N 20/00; G06N 7/005; G16H 10/60; G16H 50/70; A61B 5/02055; G16H 50/30; A61B 5/024; A61B 5/0816; A61B 5/14542; A61B 5/14532; A61B 5/163; A61B 5/4824; A61B 5/021

(57) ABSTRACT

Methods and apparatus for classifying subjects based on time series phenotypic data are disclosed. In one arrangement, a data receiving unit receives a set of first subject-data-units, each first subject-data-unit in the set comprising time series data representing phenotypic information about a different respective one of a plurality of subjects to be classified. A data processing unit processes the set of first subject-data-units to reduce a dimensionality of each first subject-data-unit, thereby obtaining a corresponding set of second subject-data-units having lower dimensionality than the first subject-data-units. The set of second subject-data-units is processed to cluster the second subject-data-units into a plurality of clusters. Each of one or more of the subjects is classified by determining to which cluster a second subject-data-unit corresponding to the subject belongs. The clustering comprises fitting a mean trajectory with error bounds to the time series data of each second subject-data-unit and clustering the resulting fitted mean trajectories with error bounds.

Embodiments of the disclosure relate to tools for classifying human or animal subjects according to phenotypic information about the subject (e.g. derived from physiological measurements such as vital sign measurements or from other information sources). The classification can be used to aid effective selection of treatment plans or to more efficiently detect heightened risk of adverse medical events or abnormalities.

Various approaches are known for classifying the health of a subject using measurements of vital signs, but most of them are either heuristic [1] or assume that data is time invariant and independent [2]. Furthermore, for modelling time-series and the state of a subject, vital sign measurements commonly contain different numbers of observations, and at irregularly sampled times. To address these problems, Gaussian processes have been used for modelling physiological time series data [3], but further improvements are desirable.

It is an object of the invention to provide improved methods and apparatus for classifying patients.

According to an aspect of the invention, there is provided a computer-implemented method of classifying subjects based on time series phenotypic data, comprising: receiving a set of first subject-data-units, each first subject-data-unit in the set comprising time series data representing phenotypic information about a different respective one of a plurality of subjects to be classified; processing the set of first subject-data-units to reduce a dimensionality of each first subject-data-unit, thereby obtaining a corresponding set of second subject-data-units having lower dimensionality than the first subject-data-units; processing the set of second subject-data-units to cluster the second subject-data-units into a plurality of clusters; and classifying each of one or more of the subjects by determining to which cluster a second subject-data-unit corresponding to the subject belongs, wherein: the clustering of the second subject-data-units comprises fitting a mean trajectory with error bounds to the time series data of each second subject-data-unit and clustering the resulting fitted mean trajectories with error bounds.

Thus, a method is provided that clusters mean trajectories with error bounds (e.g. Gaussian processes) fitted to dimension-reduced time series data. This approach has been found to provide effective clustering in situations where alternative techniques have been found to perform sub-optimally. In particular, the approach allows proper account to be taken of time dependence within time series data, as well as being able to deal effectively with missing values in the time series data. The dimension reduction processing allows the clustering to be performed even where time series are long and/or where there are many subjects. The clustering allows subjects to be classified in order to stratify risks or to phenotype patients in a population who share similar morbidity, intervention/treatment progression, or general health status.

In an embodiment, the reduction of dimensionality of the first subject-data-units is performed using a Gaussian process latent variable model. The inventors have found that this method of reducing dimensionality allows particularly effective clustering of the resulting second subject-data-units.

In an embodiment, the time series data of each first subject-data-unit is defined relative to a first set of reference time points (which may be nominally the same for all of the first subject-data-units, apart from missing values), each of one or more of the first subject-data-units as received comprises one or more missing values, and each of one or more of the first subject-data-units is processed to correct for one or more of the missing values. The inventors have found that correcting missing values in this way can be done efficiently and improves the overall clustering performance.

According to an alternative aspect, there is provided an apparatus for classifying subjects based on time series phenotypic data, comprising: a data receiving unit configured to receive a set of first subject-data-units, each first subject-data-unit in the set comprising time series data representing phenotypic information about a different respective one of a plurality of subjects to be classified; and a data processing unit configured to: process the set of first subject-data-units to reduce a dimensionality of each first subject-data-unit, thereby obtaining a corresponding set of second subject-data-units having lower dimensionality than the first subject-data-units; process the set of second subject-data-units to cluster the second subject-data-units into a plurality of clusters; and classify each of one or more of the subjects by determining to which cluster a second subject-data-unit corresponding to the subject belongs, wherein: the clustering of the second subject-data-units comprises fitting a mean trajectory with error bounds to the time series data of each second subject-data-unit and clustering the resulting fitted mean trajectories with error bounds.

Embodiments of the invention will now be described, by way of example only, with reference to the accompanying drawings in which corresponding reference symbols indicate corresponding parts, and in which:

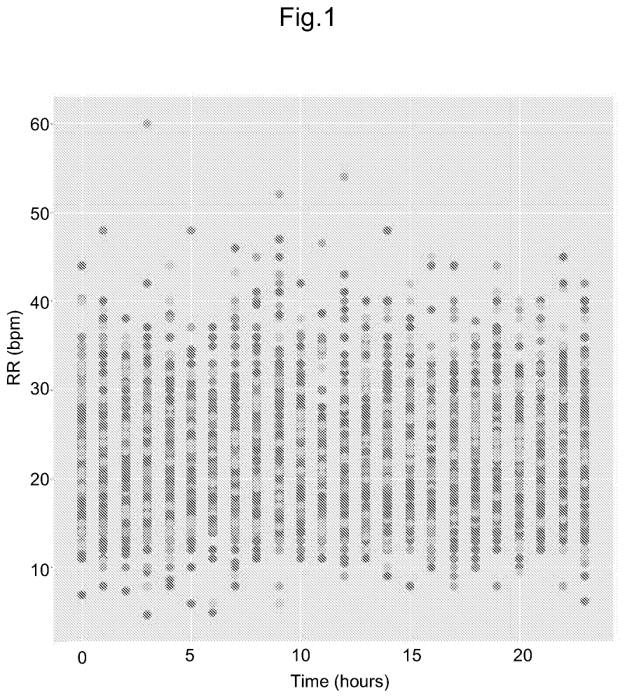

FIG. 1 depicts raw time series data comprising respiratory rate (RR) measurements (in bpm) at 24 hourly time points for a cohort of 3,385 Chronic Obstructive Pulmonary Disease (COPD) patients;

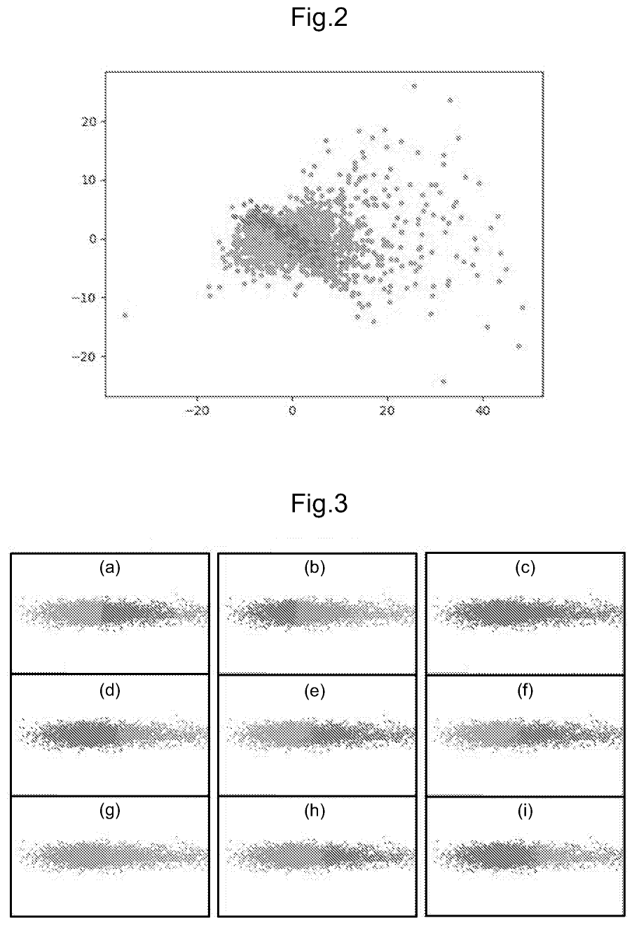

FIG. 2 depicts the result of applying a Gaussian Mixture Model (GMM) to the time series data of FIG. 1 to perform clustering (each point represents a different subject and the different shading represents different clusters);

FIGS. 3(a)-(i) depict the result of applying different clustering methods to time series data obtained by reducing the dimension of the time series data of FIG. 1, in which each point represents a different subject and the different shading represents different clusters, and: (a) shows use of Mini Batch K-Means (MiniBatchKMeans)[4], (b) shows use of Affinity Propagation (AffinityPropagation)[5], (c) shows use of Mean Shift (MeanShift)[6], (d) shows use of Spectral Clustering (SpectralClustering)[7], (e) shows use of Ward hierarchical clustering (Ward)[5], (f) shows use of Agglomerative clustering (AgglomerativeClustering)[5], (g) shows use of Balanced Iterative Reducing and Clustering using Hierarchies (Birch)[8], (h) shows use of Gaussian Mixture Model (GMM)[5], and (i) shows use of Variational Bayesian Gaussian Mixture (BayesianGMM)[5];

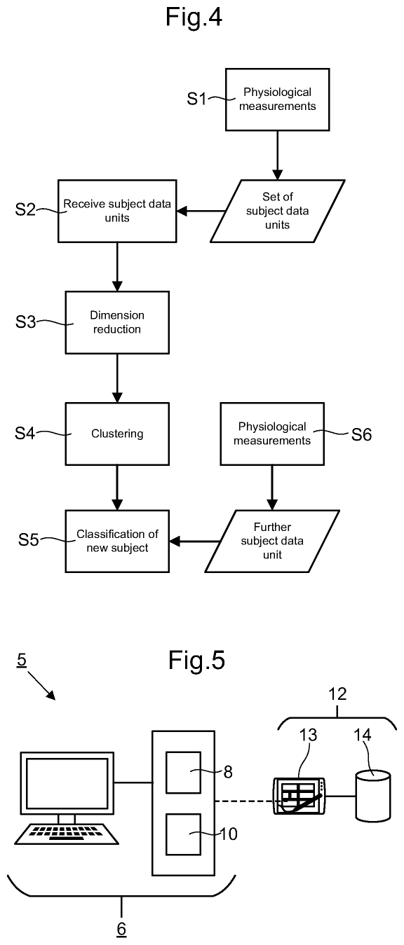

FIG. 4 depicts a method of classifying/clustering subjects based on time series phenotypic data according to an embodiment;

FIG. 5 depicts an apparatus for implementing methods of the type depicted in FIG. 4; and

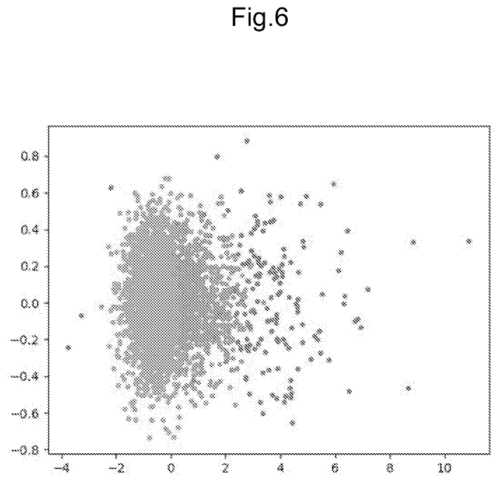

FIG. 6 depicts example results from clustering second subject-data-units according to an embodiment, in which each point represents a different subject and the different shading represents different clusters.

Methods of the present disclosure are computer-implemented. Each step of the disclosed methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g. smart TV), etc. Alternatively, the computer may consist of a distributed computing system having plural different computers connected to each other via a network such as the internet or an intranet.

FIGS. 1-3 illustrate the results of comparative methods for clustering subjects applied to a cohort of 3,385 Chronic Obstructive Pulmonary Disease (COPD) patients from which respiratory rate (RR) measurements have been obtained over 24 hours. One time point is defined for each hour so that a maximum of 24 RR data points will be provided for each subject. In practice, data will be missing at some of the time points for at least some of the patients, so that fewer than 24 RR data points may be available for those patients.

FIG. 1 shows raw time series data of the measured RR for all of the 3,385 subjects over the 24 hours. Each circular data point represents one measurement of RR from one subject. It is observed that it is impossible to separate the subjects directly using traditional clustering methods applied directly to the time series because the RRs are too close to each other.

FIG. 2 shows the result of applying a Gaussian Mixture Model (GMM) to the time series data of FIG. 1 after processing of the time series data of FIG. 1 to compute missing values (using the mean population). It is observed that the three clusters identified (denoted by three different grey shades) are not separable.

FIG. 3 shows multiple subplots of results using traditional clustering methods on the time series data of FIG. 1 after the time series data has been processed by a Gaussian process latent variable model (GPLVM) to provide dimension-reduced time series. Data points corresponding to different clusters are indicated (labelled) by different grey shades. In this example, the dimension of each time series was reduced from a maximum of 24 to 6 using the GPLVM. While some methods were able to cluster three components, their cluster labels are not accurate. In particular, only MiniBatchKMeans (FIG. 3(a)) and AffinityPropagation (FIG. 3(b)) were able to provide the correct number of cluster components, where MiniBatchKMeans would require user to pre-define the number of cluster component a priori, and AffinityPropagation produced the wrong assignments of labels for each cluster.

Embodiments of the disclosure are now discussed which provide improved performance relative to the prior art and the comparative approaches discussed above.

FIG. 4 depicts a framework for a method of classifying subjects (e.g. human or animal subjects) based on time series phenotypic data (e.g. data relating to any observable characteristic of the subject obtained at different times over a time interval). The method may be performed by an apparatus 5 as depicted in FIG. 5. The terms “human or animal subject” or “subject” may be used interchangeably with the term “patient” in the following description.

In an embodiment, the method comprises a step S1 of performing physiological measurements on a subject in a measurement session. The step S1 may generate at least a portion of phenotypic information represented by one or more first subject-data-units (discussed in further detail below). The physiological measurements may be performed used a sensor system 12 as depicted schematically in FIG. 5. The sensor system 12 may comprise a local electronic unit 13 (e.g. a tablet computer, smart phone, smart watch, etc.) and a sensor unit 14 (e.g. a blood pressure monitor, heart rate monitor, etc.). The physiological measurements may comprise one or more of the following: heart rate, respiratory rate, temperature, blood oxygenation, systolic blood pressure, diastolic blood pressure, electrocardiogram, blood glucose, temperature, blood constituent levels, pupil size, pain score, and Glasgow coma score. Alternatively or additionally, at least a portion of the phenotypic information represented by the one or more first subject-data-units may be provided by other means, such as via lab-based studies, medical imaging equipment, or manual entries made by a clinician or by the subject themselves. The phenotypic information may alternatively or additionally include one or more of the following: one or more parameters taken from a medical image, one or more parameters taken from a sample taken from the subject (e.g. blood), genetic information, and clinical information.

In step S2, the set of first subject-data-units are received by a data receiving unit 8. The data receiving unit 8 may form part of a computing system 6 (e.g. laptop computer, desktop computer, etc.). The computing system 6 may further comprise a data processing unit 10 configured to carry out steps of the method.

In some embodiments, each first subject-data-unit comprises time series data representing phenotypic information about a different respective one of a plurality of subjects to be classified. Thus, in such embodiments, the method receives one first subject-data-unit for each subject to be classified/clustered. The method can be extended so that additional subject-data-units are provided, such that plural subject-data-units are provided for each of one or more of the subjects. In cases where multiple subject-data-units are provided for a given subject, the different subject-data-units may represent physiological information obtained under different circumstances, for example during different visits of the patient or while the patient is in a different known medical condition (e.g. before and after an operation or adverse medical event).

In step S3, the set of first subject-data-units is processed to reduce a dimensionality of each first subject-data-unit. A corresponding set of second subject-data-units having lower dimensionality than the first subject-data-units is thereby obtained. The correspondence between the first subject-data-units and the second subject-data-units may be a one-to-one correspondence. Further details about how the dimensionality is defined and reduced is provided below.

In step S4, the set of second subject-data-units are processed to cluster the second subject-data-units into a plurality of clusters. Each of one or more of the subjects can then be classified (also referred to as grouped, clustered or subtyped) by determining to which cluster a second subject-data-unit corresponding to the subject belongs. Subjects that are identified as belonging to the same cluster may have characteristics in common, which enables management of those subjects to be performed more effectively (e.g. risk management, selection of treatment plan, etc.).

In an embodiment, the clustering in step S4 comprises fitting a mean trajectory with error bounds to the time series data of each second subject-data-unit and clustering the resulting fitted mean trajectories with error bounds. The fitting of the mean trajectory with error bounds to the time series data may for example comprise fitting a Gaussian process to the time series data (fitting a Gaussian process is an example of fitting a mean trajectory with error bounds).

The inventors have found the approach of steps S1-S4 allows the clustering process to be performed more reliably than alternative techniques (such as those discussed above with reference to FIGS. 1-3 or prior art techniques). The approach allows time dependence within the time series to be considered effectively, whilst also allowing missing values to be handled effectively.

In an embodiment, the clustering in step S4 uses Dirichlet Processes. The Dirichlet Processes define the number of clusters required. The Dirichlet Processes may further define which clusters the second subject-data-units belong to. In an embodiment, the Dirichlet Processes define which clusters the second subject-data-units belong to using a stick-breaking process (see [10]). In [10], a Gaussian process clustering method is performed in which a direct estimation of a mixture of Gaussian processes on time series using Dirichlet Processes (DPGP) is used in the context of analysing genetic gene expression data. The approach is effective for certain types of genetic data but there would be a dimensionality problem were the DPGP approach of [10] to be applied directly to time series of the type considered in the present disclosure that are too long and/or where there are too many subjects to be clustered. The DPGP approach of [10] also cannot deal with missing values in a robust manner. The processing occurring before the clustering step S4 according to embodiments of the present disclosure (including in particular the dimension reduction of step S3) allows the clustering to perform efficiently even for long time series and/or many subjects to be processed.

In an embodiment, the reduction of dimensionality in step S3 is configured to take account of time-dependency within each first subject-data-unit (i.e. to take account of data values at different time points in time series data being dependent on each other).

In an embodiment, the reduction of dimensionality of the first subject-data-units is performed using a Gaussian process latent variable model (GPLVM). In an embodiment, the Gaussian process latent variable model comprises a Bayesian Gaussian process latent variable model, a variational Bayesian Gaussian process latent variable model, or a hierarchical Gaussian process latent variable model. Any of the various implementations of GPLVMs known to the skilled person in the art may be used, including for example as described in [9].

In an embodiment, the time series data of each first subject-data-unit is defined relative to a first set of reference time points. In an embodiment, the first set of reference time points are nominally the same for all of the first subject-data-units. For example, in the case of the 24 hour RR data depicted in FIG. 1, the time series may be nominally defined by a set of 24 time points, although in practice data may be missing at some of the time points (e.g. where data was not collected or not collected with sufficient accuracy). Each first subject-data-unit in that example consists of a time series of 24 RR measurements at evenly spaced hourly time points. In embodiments of this type, the dimensionality of each first subject-data-unit may include at least one dimension (e.g. exactly one dimension) for each of the time points in the first set of reference time points and the reduction of dimensionality results in the time series of each second subject-data unit being defined relative to a second set of reference time points comprising fewer reference time points than the first set of reference time points. The second set of reference time points may be the same for all of the second subject-data-units. This type of dimension reduction was achieved by the data processing described above with reference to FIG. 3, in which the number of time points was reduced from a maximum of 24 to 6. Each time point may be associated with a plurality of different data values (e.g. measurements of plural different parameters, such as different physiological measurements) which may not be reduced in number by the dimension reductions. Thus, for example, in a case where 24 breathing rate (BR) measurements and 22 RR measurements were recorded (at each of 24 different time points) for each of 100 subjects, dimension reduction processing of the type discussed above could mean that the same set of time series are represented by fewer than 24 different BR and RR values. For example, the information could be represented by 12 BR values and 12 RR values for each of the 100 subjects. In this case the dimension reduction algorithm, for example the GPLVM, learns the joint relationship between the RR and the BR rather than treating them as independent of each other.

The time series data of each first subject-data-unit may take various forms. In an embodiment, data (e.g. one or more numerical values) representing one or more items of phenotypic information are provided at each of two or more of the time points, optionally including one or more of the following: a blood pressure measurement, a heart rate measurement, a breathing rate measurement, a temperature measurement, an oxygen saturation measurement. In some embodiments, data representing an error bound of an item of phenotypic information is provided at each of two or more of the time points in each of the first subject-data-units. The time series data may comprise evenly sampled data (i.e. data values at time points that are spaced apart evenly) or unevenly sample data. In an embodiment, each of one or more of the first subject-data-units as received comprises one or more missing values, wherein each missing value is defined as the absence of an expected item of phenotypic information at one or more of the time points in the reference set of time points. The time series data may comprise nominally evenly sampled data but with missing values.

In an embodiment, first subject-data-units as received initially in step S2 are processed to improve their quality before being used in step S3. For example, unevenly sampled data may be processed (e.g. using interpolation and/or averaging) to provide evenly sampled data. In an embodiment, each of one or more of the first subject-data-units is processed to correct for one or more missing values. In an embodiment, the correction for each missing value comprises inserting a mathematically generated value at the time point corresponding to the missing value. In an embodiment, the mathematically generated value is generated based on phenotypic information obtained about the same subject at a different time or based on phenotypic information obtained about one or more other subjects. In an embodiment, the mathematically generated value is generated based on a mean trajectory with error bounds (e.g. a Gaussian process) fitted to a first subject-data-unit.

In an embodiment, a further step S5 is provided in which a further first subject-data-unit is obtained. The further first subject-data-unit comprises time series data representing phenotypic information about a further subject. The further first subject-data-unit may take any of the forms described above for the other first subject-data-units. In an embodiment, the further first subject-data-unit is at least partially obtained by performing one or more physiological measurements on the further subject (step S6).

Step S5 further comprises processing the further first subject-data-unit to reduce a dimensionality of the further first subject-data-unit and thereby obtain a further second subject-data-unit. The processing to reduce the dimensionality may be performed using any of the approaches described above for reducing the dimensionality of the first subject-data-units.

Step S5 further comprises classifying the further subject by determining to which of the clusters the further second subject-data-unit belongs.

Thus, steps S2-S4 of the method effectively train the method by generating clusters of the second subject-data-units. A first subject-data-unit from a new subject can then be processed to generate a second subject-data-unit that can be compared with the clusters to classify the new subject.

FIG. 6 shows example results from an embodiment. In this example, first subject-data-units were dimensionally reduced and then clustered using Gaussian Process with Dirichlet Process. The number of clusters was obtained in an unsupervised manner (i.e., without the need to predefine the number of clusters, which is a common problem in clustering methods). In contrast to the comparative example discussed above with reference to FIG. 2, three clusters (denoted by three different grey shades) are identified and are well separable from each other.

### REFERENCES

[1] H. Gao, A. McDonnell, D. A. Harrison, T. Moore, S. Adam, K. Daly, L. Esmonde, D. R. Goldhill, G. J. Parry, A. Rashidian et al., “Systematic review and evaluation of physiological track and trigger warning systems for identifying at-risk patients on the ward,” Intensive care medicine, vol. 33, no. 4, pp. 667-679, 2007.
[2] L. Tarassenko, A. Hann, and D. Young, “Integrated monitoring and analysis for early warning of patient deterioration,” British Journal of Anaesthesia, vol. 97, no. 1, pp. 64-68, 2006.
[3] L. Clifton, D. A. Clifton, M. A. Pimentel, P. J. Watkinson, and L. Tarassenko, “Gaussian processes for personalized e-health monitoring with wearable sensors,” IEEE Transactions on Biomedical Engineering, vol. 60, no. 1, pp. 193-197, 2013.
[4] D. Sculley, “Web Scale K-Means clustering”, Proceedings of the 19th international conference on World wide web, 2010.
[5] Pedregosa et al., “Scikit-learn: Machine Learning in Python”, Journal of Machine Learning Research 12, pp. 2825-2830, 2011,
[6] D. Comaniciu and P. Meer,“Mean shift: A robust approach toward feature space analysis.”, IEEE Transactions on Pattern Analysis and Machine Intelligence, 2002.
[7] Andrew Y. Ng, Michael 1. Jordan, Pair Weiss, “On Spectral Clustering: Analysis and an algorithm”, 2001,
[8] Strehl, Alexander, and Joydeep Ghosh, “Cluster ensembles—a knowledge reuse framework for combining multiple partitions”, Journal of Machine Learning Research 3: 583-617, 2002.
[9] N. D. Lawrence, “Gaussian Process Latent Variable Models for Visualisation of High Dimensional Data,” Advances in neural information processing systems, 2004.
[10] J. Hensman, M. Rattray, N. D. Lawrence, “Fast Nonparametric Clustering of Structured Time-Series,” IEEE Transactions on Pattern Analysis and Machine Intelligence, Vol. 37, No. 2, 383-393, February 2015.

1. A computer-implemented method of classifying subjects based on time series phenotypic data, comprising:
receiving a set of first subject-data-units, each first subject-data-unit in the set comprising time series data representing phenotypic information about a different respective one of a plurality of subjects to be classified;
processing the set of first subject-data-units to reduce a dimensionality of each first subject-data-unit, thereby obtaining a corresponding set of second subject-data-units having lower dimensionality than the first subject-data-units;
processing the set of second subject-data-units to cluster the second subject-data-units into a plurality of clusters; and
classifying each of one or more of the subjects by determining to which cluster a second subject-data-unit corresponding to the subject belongs, wherein:
the clustering of the second subject-data-units comprises fitting a mean trajectory with error bounds to the time series data of each second subject-data-unit and clustering the resulting fitted mean trajectories with error bounds.

2. The method of claim 1, wherein the fitting of the mean trajectory with error bounds to the time series data comprises fitting a Gaussian process to the time series data.

3. The method of claim 1, wherein the clustering of the fitted mean trajectories with error bounds uses Dirichlet Processes, where the Dirichlet Processes define the number of clusters required.

4. The method of claim 3, wherein the Dirichlet Processes assign the second subject-data-units to the clusters.

5. The method of claim 4, wherein the Dirichlet Processes assign the second subject-data-units to the clusters using a stick-breaking process.

6. The method of claim 1, wherein the reduction of dimensionality of the first subject-data-units takes account of time-dependency within each first subject-data-unit.

7. The method of claim 6, wherein the reduction of dimensionality of the first subject-data-units is performed using a Gaussian process latent variable model.

8. The method of claim 7, wherein the Gaussian process latent variable model comprises a Bayesian Gaussian process latent variable model, a variational Bayesian Gaussian process latent variable model, or a hierarchical Gaussian process latent variable model.

9. The method of claim 1, wherein the time series data of each first subject-data-unit is defined relative to a first set of reference time points.

10. The method of claim 9, wherein the dimensionality of each first subject-data-unit includes at least one dimension for each of the time points in the first set of reference time points and the reduction of dimensionality results in the time series of each second subject-data-unit being defined relative to a second set of reference time points comprising fewer reference time points than the first set of reference time points.

11. The method of claim 9, wherein the set of reference time points comprises a plurality of time points spaced apart from each other by a constant time interval.

12. The method of claim 9, wherein data representing each of one or more of the following is provided at each of two or more of the time points: heart rate, respiratory rate, temperature, blood oxygenation, systolic blood pressure, diastolic blood pressure, electrocardiogram, blood glucose, temperature, blood constituent levels, pupil size, pain score, Glasgow coma score or any measurements performed on a sample from the human or animal.

13. The method of claim 9, wherein each of one or more of the first subject-data-units as received comprises one or more missing values, each missing value being defined as the absence of an expected item of phenotypic information at one or more of the time points.

14. The method of claim 13, wherein each of one or more of the first subject-data-units is processed to correct for one or more of the missing values.

15. The method of claim 14, wherein the correction for each missing value comprises inserting a mathematically generated value at the time point corresponding to the missing value.

16. The method of claim 15, wherein the mathematically generated value is generated based on phenotypic information obtained about the same subject at a different time or based on phenotypic information obtained about one or more other subjects.

17. The method of claim 15, wherein the mathematically generated value is generated based on a mean trajectory with error bounds fitted to a first subject-data-unit.

18. The method of claim 9, wherein data representing an error bound of an item of phenotypic information is provided at each of two or more of the time points in each of the first subject-data-units.

19. The method of claim 1, comprising:
obtaining a further first subject-data-unit comprising time series data representing phenotypic information about a further subject;
processing the further first subject-data-unit to reduce a dimensionality of the further first subject-data-unit and thereby obtain a further second subject-data-unit; and
classifying the further subject by determining to which of the clusters the further second subject-data-unit belongs.

20. The method of claim 1, further comprising:
performing physiological measurements to generate at least a portion of the phenotypic information represented by one or more of the first subject-data-units.

21. A computer program comprising computer-readable instructions that cause a computer to perform the method of claim 1.

22. A computer program product storing the computer program of claim 21.

23. An apparatus for classifying subjects based on time series phenotypic data, comprising:
a data receiving unit configured to receive a set of first subject-data-units, each first subject-data-unit in the set comprising time series data representing phenotypic information about a different respective one of a plurality of subjects to be classified; and
a data processing unit configured to:
process the set of first subject-data-units to reduce a dimensionality of each first subject-data-unit, thereby obtaining a corresponding set of second subject-data-units having lower dimensionality than the first subject-data-units;
process the set of second subject-data-units to cluster the second subject-data-units into a plurality of clusters; and
classify each of one or more of the subjects by determining to which cluster a second subject-data-unit corresponding to the subject belongs, wherein:
the clustering of the second subject-data-units comprises fitting a mean trajectory with error bounds to the time series data of each second subject-data-unit and clustering the resulting fitted mean trajectories with error bounds.

24. The device of claim 23, further comprising a sensor system configured to perform physiological measurements on a subject to provide a subject-data-unit comprising time series data representing phenotypic information about the subject.
